(19) United States
(12) Patent Application Publication
(10) Pub. No.: US 2008/0249183 A1
(43) Pub. Date: Oct. 9, 2008

(54) Treatment of androgen-deprivation induced osteoporosis

(76) Inventors: Mitchell S. Steiner, Germantown, TN (US); Karen A. Veverka, Cordova, TN (US)

Correspondence Address: Pearl Cohen Zedek Latzer, LLP, 1500 Broadway, 12th Floor, New York, NY, 10036, US

(21) Appl. No.: 12/071,591

(22) Filed: Feb. 22, 2008

Related U.S. Application Data
(63) Continuation-in-part of application No. 11/328,393, filed on Jan. 9, 2006.
Application No. 11/328,393 is a continuation-in-part of application No. 10/944,465, filed on Sep. 20, 2004.
Application No. 10/944,465 is a continuation-in-part of application No. 10/778,334, filed on Feb. 17, 2004.
Application No. 10/778,334 is a continuation-in-part of application No. 10/609,684, filed on Jul. 1, 2003, now abandoned.
Application No. 10/609,684 is a continuation-in-part of application No. 10/305,363, filed on Nov. 27, 2002, now Pat. No. 6,899,888.
(60) Provisional application No. 60/333,734, filed on Nov. 29, 2001.

(51) Int. Cl. A61K 31/138; A61P 19/10; A61P 5/26

(52) U.S. Cl. 514/648

(57) ABSTRACT

This invention provides a method of treating androgen-deprivation induced osteoporosis, bone fractures or loss of bone mineral density (BMD) in a male human subject suffering from prostate cancer, wherein the subject has a precipitous decline in androgen levels, by administering a pharmaceutical composition comprising Toremifene or its analog, derivative, isomer, metabolite, pharmaceutically acceptable salt, pharmaceutical product, hydrate, N-oxide, or any combination thereof, to the subject, wherein the method increases bone density without increasing androgen and specifically testosterone levels in the subject.

### CROSS REFERENCE TO RELATED APPLICATIONS

This application is a continuation-in-part of U.S. application Ser. No. 11/329,393, filed Jan. 11, 2006, which is a continuation-in-part of U.S. application Ser. No. 10/944,465, filed Sep. 20, 2004, which is a continuation-in-part of U.S. application Ser. No. 10/778,334, filed Feb. 17, 2004, which is a continuation-in-part of U.S. application Ser. No. 10/609,684, filed Jul. 3, 2003, which is a continuation-in-part of U.S. application Ser. No. 10/305,363, filed Nov. 27, 2002, and claims priority of U.S. Provisional Application Ser. No. 60/333,734, filed Nov. 29, 2001, the contents of which are specifically incorporated herein by reference.

### FIELD OF INVENTION

This invention relates to the prevention and treatment of androgen-deprivation therapy (ADT) induced bone diseases or conditions in men suffering from prostate cancer via the administration of a selective estrogen receptor modulator without increasing testosterone levels in the subject. In other aspects, this invention relates to a method of treating, preventing, suppressing, inhibiting, or reducing the risk of developing ADT-induced osteoporosis, bone fractures, and/or loss of bone mineral density (BMD) in men suffering from prostate cancer, comprising administering to a male subject suffering from prostate cancer an anti-estrogen agent, a selective estrogen receptor modulator agent, or a triphenylethylene and/or its analog, derivative, isomer, metabolite, pharmaceutically acceptable salt, pharmaceutical product, hydrate, N-oxide, or any combination thereof.

### BACKGROUND OF THE INVENTION

The relative bone mineral density of males decreases with age. Decreased bone mineral content and density correlates with decreased bone strength and predisposes the bone to fracture. Sex-hormones appear to play a role in bone homeostasis, with physiologic concentrations of androgens and estrogens being involved in maintaining bone health, throughout adult life. Changes in sex hormone levels is associated with an increase in the rate of bone remodeling, skewing the normal balance between bone resorption and formation to favor resorption, contributing to an overall loss of bone mass.

Prostate cancer is one of the most frequently diagnosed noncutaneous cancers among men in the United States. One of the approaches to the treatment of prostate cancer is androgen deprivation therapy (ADT) in the subject. The male sex hormone, testosterone, stimulates the growth of cancerous prostatic cells and, therefore, is the primary fuel for the growth of prostate cancer. The goal of ADT is to decrease stimulation of cancerous prostatic cells by testosterone. Testosterone is normally produced by the testes in response to stimulation from a hormonal signal called luteinizing hormone (LH) which in turn is stimulated by luteinizing-hormone releasing hormone (LH-RH). Androgen deprivation therapy in male subjects has been accomplished surgically, by bilateral orchidectomy, and/or chemically, for example, via the administration of LH-RH agonists (LHRHα) and/or antiandrogens.

Androgen deprivation therapy in patients with micrometastatic disease has been shown to prolong survival [Messing E M, et al (1999), N Engl J Med 34, 1781-1788; Newling (2001), Urology 58(Suppl 2A), 50-55]. Moreover, ADT is being employed in numerous new clinical settings, including neoadjuvant therapy prior to radical prostatectomy, long-term adjuvant therapy for patients at high risk for recurrence following radiation or surgery, neoadjuvant therapy for radiation, and treatment of biochemical recurrence following radiation or surgery [Carroll, et al (2001), Urology 58, 1-4; Horwitz E M, et al (2001), Int J Radiat Oncol Biol Phy Mar 15; 49(4), 947-56]. Thus, more prostate cancer patients have become candidates for and are being treated by androgen ablation, and at an earlier time and for a prolonged period of time, than previously undertaken. Treatment lasting 10 or more years with ADT is not uncommon.

Unfortunately, androgen deprivation therapy is accompanied by significant side effects, including hot flashes, gynecomastia, osteoporosis, decreased lean muscle mass, depression and other mood changes, loss of libido, and erectile dysfunction [Stege R (2000), Prostate Suppl 10, 38-42]. Consequently, complications of androgen blockade now contribute significantly to the morbidity and in some cases the mortality, of men suffering from prostate cancer.

In males, the natural decline in sex-hormones at maturity (direct decline in androgens as well as lower levels of estrogens derived from peripheral aromatization of androgens) is associated with the frailty of bones. The clinical course of bone disease in males who have undergone androgen deprivation therapy differs from maturity onset bone disease, both in terms of the rapidity of bone loss, and amount of loss over a brief period of time. A precipitous drop in bone density occurs in males who have undergone androgen deprivation therapy.

The serum concentration of testosterone decreases with age, reportedly at a rate of 0.2 to 0.4% a year in normal healthy males. The age-related decline in testosterone, also termed Male Menopause or Andropause, is a gradual decline in serum testosterone, in marked contrast to the rapid, significant decline evident in patients undergoing ADT.

Given that more patients today are being treated by long-term androgen deprivation, ADT-induced osteoporosis has become a clinically important side effect in men suffering from prostate cancer and undergoing androgen deprivation therapy. Loss of bone mineral density (BMD) occurs in the majority of patients being treated by androgen deprivation by 6 months. New innovative approaches are urgently needed to decrease the incidence of ADT-induced osteoporosis and bone disease in men suffering from prostate cancer.

### SUMMARY OF THE INVENTION

In one embodiment, this invention provides a method of treating androgen-deprivation therapy induced osteoporosis in a male human subject suffering from prostate cancer, said method comprising the step of administering 80 mg per day of Compound I:

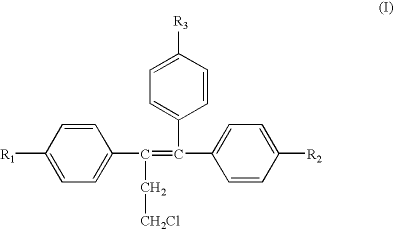

wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms, or a pharmaceutically acceptable salt thereof to said subject, wherein said method increases bone density without increasing androgen levels or in some embodiments, specifically testosterone levels in the subject in a testosterone-independent manner, thereby treating androgen deprivation therapy (ADT)-induced osteoporosis in a male human subject suffering from prostate cancer.

In one embodiment, this invention provides method of preventing ADT-induced osteoporosis in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby preventing ADT-induced osteoporosis in a male human subject suffering from prostate cancer.

In one embodiment, this invention provides a method of suppressing, inhibiting or reducing the risk of developing ADT-induced osteoporosis in a male human subject suffering from prostate cancer, said method comprising the step of administering 80 mg per day of Compound I or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced osteoporosis in a male human subject suffering from prostate cancer.

In one embodiment, this invention provides a method of treating ADT-induced loss of bone mineral density (BMD) in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby treating ADT-induced loss of bone mineral density (BMD) in a male human subject suffering from prostate cancer.

In one embodiment, this invention provides a method of preventing ADT-induced loss of bone mineral density (BMD) in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby preventing ADT-induced loss of bone mineral density (BMD) in a male human subject suffering from prostate cancer.

In one embodiment, this invention provides a method of suppressing, inhibiting or reducing the risk of developing ADT-induced loss of bone mineral density (BMD) in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced loss of bone mineral density (BMD) in a male human subject suffering from prostate cancer.

In one embodiment, this invention provides a method of treating ADT-induced bone fractures in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby treating ADT-induced bone fractures in a male human subject suffering from prostate cancer.

In one embodiment, this invention provides a method of preventing ADT-induced bone fractures in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby preventing ADT-induced bone fractures in a male human subject suffering from prostate cancer.

In one embodiment, this invention provides a method of suppressing, inhibiting or reducing the risk of developing ADT-induced bone fractures in a male human subject suffering from prostate cancer, said method comprising the step of administering 80 mg per day of Compound I, or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced bone fractures in a male human subject suffering from prostate cancer.

In one embodiment, this invention provides a method of treating androgen-deprivation therapy induced osteoporosis in a male human subject suffering from prostate cancer, said method comprising the step of administering Compound I:

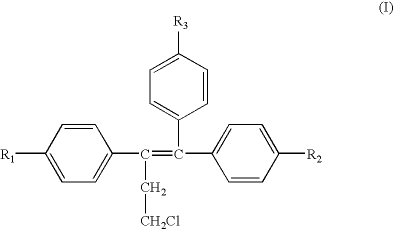

wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms, or a pharmaceutically acceptable salt thereof to said subject, wherein said method increases bone density without increasing androgen levels or in some embodiments, specifically testosterone levels in the subject in a testosterone-independent manner, thereby treating androgen deprivation therapy (ADT)-induced osteoporosis in a male human subject suffering from prostate cancer.

In one embodiment, this invention provides method of preventing ADT-induced osteoporosis in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby preventing ADT-induced osteoporosis in a male human subject suffering from prostate cancer.

In one embodiment, this invention provides a method of suppressing, inhibiting or reducing the risk of developing ADT-induced osteoporosis in a male human subject suffering from prostate cancer, said method comprising the step of administering Compound I or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced osteoporosis in a male human subject suffering from prostate cancer.

In one embodiment, this invention provides a method of treating ADT-induced loss of bone mineral density (BMD) in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby treating ADT-induced loss of bone mineral density (BMD) in a male human subject suffering from prostate cancer.

In one embodiment, this invention provides a method of preventing ADT-induced loss of bone mineral density (BMD) in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby preventing ADT-induced loss of bone mineral density (BMD) in a male human subject suffering from prostate cancer.

In one embodiment, this invention provides a method of suppressing, inhibiting or reducing the risk of developing ADT-induced loss of bone mineral density (BMD) in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced loss of bone mineral density (BMD) in a male human subject suffering from prostate cancer.

In one embodiment, this invention provides a method of treating ADT-induced bone fractures in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby treating ADT-induced bone fractures in a male human subject suffering from prostate cancer.

In one embodiment, this invention provides a method of preventing ADT-induced bone fractures in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby preventing ADT-induced bone fractures in a male human subject suffering from prostate cancer.

In one embodiment, this invention provides a method of suppressing, inhibiting or reducing the risk of developing ADT-induced bone fractures in a male human subject suffering from prostate cancer, said method comprising the step of administering Compound I, or a pharmaceutically acceptable salt thereof to said subject, wherein said method increases bone density without increasing androgen levels or in some embodiments, specifically testosterone levels in the subject, thereby suppressing, inhibiting or reducing the risk of developing ADT-induced bone fractures in a male human subject suffering from prostate cancer.

### DETAILED DESCRIPTION OF THE INVENTION

This invention provides, in some embodiments, methods of 1) treating ADT-induced osteoporosis; 2) preventing ADT-induced osteoporosis; 3) suppressing, inhibiting or reducing the risk of developing ADT-induced osteoporosis; 4) treating ADT-induced loss of bone mineral density (BMD); 5) preventing ADT-induced loss of bone mineral density (BMD); 6) suppressing, inhibiting or reducing the risk of developing ADT-induced loss of bone mineral density (BMD); 7) treating ADT-induced bone fractures; 8) preventing ADT-induced bone fractures; 9) suppressing, inhibiting or reducing the risk of developing ADT-induced bone fractures in a male human subject suffering from prostate cancer, by administering to the subject 80 mg per day of an antiestrogen; or in another embodiment a selective estrogen receptor modulator (SERM); or in another embodiment a triphenylethylene; or in one embodiment, Compound I, represented by the formula:

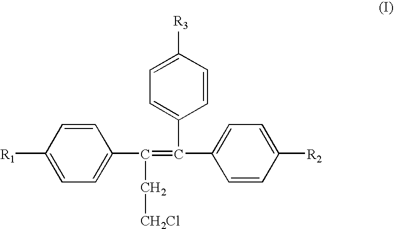

wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms, or a pharmaceutically acceptable salt thereof; or in another embodiment, Toremifene and/or its analog, derivative, isomer, metabolite, pharmaceutically acceptable salt, pharmaceutical product, hydrate, N-oxide, or any combination thereof, wherein the method, inter alia, increases bone density without increasing androgen levels or in some embodiments, specifically testosterone levels in the subject, thereby being effective in the activities of (1)-(9) listed above.

In males, the natural decline in sex-hormones at maturity (direct decline in androgens as well as lower levels of estrogens derived from peripheral aromatization of androgens) is associated with the frailty of bones. The clinical course of bone disease in males who have undergone androgen deprivation therapy differs from maturity onset bone disease, both in terms of the rapidity of bone loss, and amount of loss over a brief period of time. A precipitous drop in bone density occurs in males who have undergone androgen deprivation therapy.

The administration of Compound I, such as, for example, Toremifene, at a daily dosage of about 80 mg, was shown herein to increase bone density (Example 1). Example 2 demonstrated human clinical trial results demonstrating a highly significant reduction in bone loss and incidence of bone fractures, as a consequence of Toremifene treatment, in subjects undergoing ADT.

Toremifene citrate 80 mg demonstrated a 50% reduction in morphometric vertebral fractures (p<0.05; 5.0% fracture rate in the placebo group) in a modified intent to treat analysis, which included patients with at least one evaluable study radiograph and a minimum of one dose of study drug or placebo. In pre-specified subset analyses, among patients who were greater than 80% treatment compliant, toremifene citrate 80 mg reduced vertebral morphometric fractures by 61% (p=0.017). When patients who experienced greater than 7% bone loss at one year were considered along with new morphometric vertebral fractures as treatment failures, toremifene citrate 80 mg compared to placebo demonstrated a 56% reduction (p=0.003). The finding is surprising in that the subject has dramatic rapid bone loss as a consequence of androgen ablation, yet profound improvement following administration of toremifene citrate 80 mg, this in an environment with androgen ablation, i.e. there is no concomitant rise in circulating testosterone levels. Toremifene citrate, in a time- and dose-dependent manner, reduced the level of both free and total testosterone in a clinical trial where male human subjects undergoing ADT for prostate cancer were administered toremifene citrate over a period of 6 months, demonstrating that toremifene administration does not result in increased testosterone levels. In some embodiments, this represents a testosterone-independent mechanism for Toremifene-mediated effects on bone density, lipid profile alteration and gynecomastia, as further described hereinunder.

Patients treated with toremifene citrate 80 mg demonstrated statistically significant increases compared to placebo in bone mineral density in the lumbar spine (+2.0%; p<0.0001), and other skeletal sites (hip and femur) had similar increases (p<0.0001). Example 4 demonstrated that toremifene citrate 80 mg treatment compared to placebo also resulted in a decrease in total cholesterol (p=0.011), LDL (p=0.018), and triglycerides (p<0.0001) levels, and an increase in HDL (p=0.001). There were also statistically significant improvements in gynecomastia (p=0.003).

Accordingly, this invention provides a method of treating, preventing, reducing the incidence of, reducing the onset of, reducing the severity of, or reducing the risk of developing gynecomastia or pathologic lipid profiles in a male human subject suffering from prostate cancer, the method comprising administering 80 mg per day of Compound I or in another embodiment Toremifene, or a pharmaceutically acceptable salt thereof to said subject, wherein said treating, preventing, reducing the incidence of, reducing the onset of, reducing the severity of, or reducing the risk of developing gynecomastia is without concomitant increased androgen levels in the subject.

In some embodiments, the term “pathologic lipid profiles” refers to a serum total cholesterol concentration of greater than about 5.2 mmol/L (about 200 mg/dL), or in some embodiments, a serum LDL level above 100 mg/dl, or in some embodiments, above 130 mg/dl, or in some embodiments, above 160 mg/dl. In some embodiments, the term “pathologic lipid profiles” refers to a serum HDL level of less than 40 mg/dl. In some embodiments, the term “pathologic lipid profiles” refers to a serum triglyceride level of more than 150 mg/dl. In some embodiments, this invention provides a method to treat a pathologic lipid profile which may comprise a subject exhibiting one or more pathologic values as described herein. According to this aspect, such a subject will have prostate cancer and have been or being treated with ADT, whereupon such pathologic lipid profiles manifest as a consequence of ADT. According to this aspect, and in some embodiments, such treatment may entail administering a Compound I, which in some embodiments is Toremifene to alter pathologic lipid profiles toward healthier profiles, for example, via reduction in circulating cholesterol and triglyceride levels.

Toremifene is an example of a triphenylalkylene compound described in U.S. Pat. Nos. 4,696,949 and 5,491,173 to Toivola et al., the disclosures of which are incorporated herein by reference. Formulations containing Toremifene are described, for example, in U.S. Pat. No. 5,571,534 to Jalonen et al. and in U.S. Pat. No. 5,605,700 to DeGregorio et al., the disclosures of which are incorporated herein by reference.

In one embodiment the methods of this invention are directed to Toremifene treatment, prevention, suppression, inhibition or reduction of the risk of developing androgen-deprivation induced osteoporosis and/or loss of BMD and/or a Toremifene analog, derivative, isomer, metabolite, pharmaceutically acceptable salt, pharmaceutical product, hydrate, N-oxide, or any combination thereof in a male human subject suffering from prostate cancer.

Unexpectedly, a dose of 80 mg/day in humans was more effective in increasing bone density, as opposed to other dosages tested, in the assessed population, and in subjects without increasing testosterone levels, but rather in a subject having undergone androgen deprivation or ablation.

ADT causes a precipitous decline or decrease in bone mineral density or mass. In some embodiments, the term “precipitous decline or decrease” refers to a rapid loss over time. In some embodiments, the term “precipitous decline or decrease” is with respect to an age-matched subject or population, not undergoing ADT, or in some embodiments, with respect to an elderly subject or population, advanced in age beyond that of the population or subject undergoing ADT, yet the subject or population undergoing ADT exhibits comparable bone loss as the age-matched or elderly subjects or population, however, the latter exhibit such loss over a period of months to years, while the ADT treated subjects or populations exhibit comparable loss in a matter of weeks to months. In another embodiment, the ADT treated subjects or population exhibit much greater bone loss in the timeframe in which bone loss is evident in the age-matched or elderly subject or population. In some embodiments, the ADT treated subjects or population exhibit much greater bone loss in a much shorter timeframe in which bone loss is evident in the age-matched or elderly subject or population. In some embodiments, the precipitous decline in bone density results in a more than 5% decrease in bone density as determined by conventional techniques over a period of weeks to months, or in some embodiments, the precipitous decline in bone density results in a more than 10% decrease in bone density as determined by conventional techniques over a period of weeks to months, the precipitous decline in bone density results in a more than 15% decrease in bone density as determined by conventional techniques over a period of weeks to months, the precipitous decline in bone density results in a more than 17% decrease in bone density as determined by conventional techniques over a period of weeks to months, the precipitous decline in bone density results in a more than 20% decrease in bone density as determined by conventional techniques over a period of weeks to months, the precipitous decline in bone density results in a more than 25% decrease in bone density as determined by conventional techniques over a period of weeks to months, the precipitous decline in bone density results in a more than 30% decrease or more in bone density as determined by conventional techniques over a period of weeks to months. In some embodiments, the timeframe referred to herein is over a period of months, or a few years, which in some embodiments is less than 3 years. Subjects undergoing ADT have a precipitous decline or decrease in androgen, or in some embodiments, specifically testosterone levels. In some embodiments, the term “precipitous decline or decrease” refers to a rapid loss over time. In some embodiments, the term “precipitous decline or decrease” is with respect to an age-matched subject or population, not undergoing ADT, or in some embodiments, with respect to an elderly subject or population, advanced in age beyond that of the population or subject undergoing ADT, yet the subject or population undergoing ADT exhibits comparable decline in androgen or in some embodiments, specifically testosterone levels as the age-matched or elderly subjects or population, however, the latter exhibit such loss over a period of months to years, while the ADT treated subjects or populations exhibit comparable loss in a matter of weeks to months. In another embodiment, the ADT treated subjects or population exhibit much greater decline or relative ablation of androgens, including in some embodiments, testosterone, in the timeframe in which androgen or in some embodiments, testosterone decline or relative ablation is evident in the age-matched or elderly subject or population. In some embodiments, the ADT treated subjects or population exhibit much greater androgen, or in some embodiments, testosterone decline or relative ablation in a much shorter timeframe in which such decline or relative ablation is evident in the age-matched or elderly subject or population. In some embodiments, the precipitous decline or relative ablation in androgen or in some embodiments, testosterone results in a more than 0.5% decrease in circulating levels as determined by conventional techniques over a period of weeks to months, or in some embodiments, decline or relative ablation in androgen or in some embodiments, testosterone results in a more than 1% decrease in circulating levels as determined by conventional techniques over a period of weeks to months, or in some embodiments, decline or relative ablation in androgen or in some embodiments, testosterone results in a more than 1.5% decrease in circulating levels as determined by conventional techniques over a period of weeks to months, or in some embodiments, decline or relative ablation in androgen or in some embodiments, testosterone results in a more than 2% decrease in circulating levels as determined by conventional techniques over a period of weeks to months, or in some embodiments, decline or relative ablation in androgen or in some embodiments, testosterone results in a more than 2.5% decrease in circulating levels as determined by conventional techniques over a period of weeks to months, or in some embodiments, decline or relative ablation in androgen or in some embodiments, testosterone results in a more than 3% decrease in circulating levels as determined by conventional techniques over a period of weeks to months, or in some embodiments, decline or relative ablation in androgen or in some embodiments, testosterone results in a more than 4% decrease in circulating levels as determined by conventional techniques over a period of weeks to months, or in some embodiments, decline or relative ablation in androgen or in some embodiments, testosterone results in a more than 5% decrease in circulating levels as determined by conventional techniques over a period of weeks to months, or in some embodiments, decline or relative ablation in androgen or in some embodiments, testosterone results in a more than 10% decrease in circulating levels as determined by conventional techniques over a period of weeks to months. In some embodiments, the timeframe referred to herein is over a period of months, or a few years, which in some embodiments is less than 3 years

Elderly males typically have circulating testosterone levels of about 300 to about 800 ng/dl. In some embodiments, ADT stimulated precipitous androgen decline refers to a circulating level reduced in comparison to that of elderly males, as described hereinabove. In some embodiments, ADT stimulated precipitous androgen decline refers to a circulating testosterone level of about 20 to about 50 ng/dl, or in some embodiments, from about 20 to about 150 ng/dl.

In some embodiments, the term “precipitous decline or decrease in androgen, or testosterone levels” is to be distinguished from “relative decline or decreases in androgen or testosterone levels”, the latter of which may be suited to describe age-related decline in male subjects, as opposed to the precipitous decline as a function of the administration of specific agents which comprise conventional androgen deprivation therapy.

In some embodiments, the methods of this invention provide for treating, preventing, suppressing, inhibiting, reducing the incidence of, reducing the severity of, reducing the pathogenesis of, or reducing the risk of developing androgen-deprivation induced osteoporosis, loss of bone mineral density or bone fractures, or gynecomastia or pathologic lipid profiles in a male human subject suffering from prostate cancer, via administering a SERM, which in one embodiment is Compound I, and in one embodiment is Toremifene, at a dosage of 80 mg per day, wherein the method increases bone density, reduces gynecomastia or pain associated with gynecomastia, or alters lipid profiles such that they are less or no longer pathologic, without increasing androgen levels in the subject.

In some embodiments, the phrase “without increasing androgen levels in the subject” refers to the lack of stimulation of the antiestrogen, SERM, Compound I or Toremifene to stimulate testosterone in a subject undergoing or having undergone ADT. The term “without increasing androgen levels in the subject” refers to a relative increase, which varies from baseline testosterone levels by less than 1%, or in some embodiments, less than 5%, or in some embodiments, less than 10%, or in some embodiments, any value there-between. In some embodiments, the term “without increasing androgen levels in the subject” refers to lack of increase of testosterone in a subject undergoing ADT as compared to prior to administration of the antiestrogen, SERM, Compound I or Toremifene. In some embodiments, the term “without increasing androgen levels in the subject” refers to the subject having less than 0.5 ng/ml circulating testosterone, post administration of Toremifene, Compound I or the SERM or the antiestrogen.

In one embodiment, the methods of this invention entail administering 80 mg dosage per day, in symptomatic subjects, over a prolonged period of time. In one embodiment, the treatment is provided for 1 month, or in another embodiment, for 1-6 months, or in another embodiment, for 1-12 months, or in another embodiment, for at least one year, or in another embodiment, for the duration of androgen deprivation therapy, by chemical means. In another embodiment, the treatment is continuous, or in another embodiment, the treatment is cyclic, with specified periods of treatment and lack of treatment. In another embodiment, treatment is continued and discontinued as a function of bone density or bone mineral loss, such that the subject is evaluated at specified periods, and the administration regimen is tailored to individual responses to treatment.

The present invention provides, in some embodiments, a method of treating androgen-deprivation induced osteoporosis in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby treating androgen-deprivation induced osteoporosis in a male human subject suffering from prostate cancer.

In another embodiment, the invention provides a method of preventing androgen-deprivation induced osteoporosis in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby preventing androgen-deprivation induced osteoporosis in a male human subject suffering from prostate cancer.

In another embodiment, the present invention provides a method of suppressing, inhibiting or reducing the risk of developing androgen-deprivation induced osteoporosis in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby suppressing, inhibiting or reducing the risk of developing androgen-deprivation induced osteoporosis in a male human subject suffering from prostate cancer.

In another embodiment, the present invention provides a method of androgen-deprivation induced loss of bone mineral density (BMD) in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby treating androgen-deprivation induced loss of bone mineral density (BMD) in a male human subject suffering from prostate cancer.

In another embodiment, the present invention provides a method of preventing androgen-deprivation induced loss of bone mineral density (BMD) in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby preventing androgen-deprivation induced loss of bone mineral density (BMD) in a male human subject suffering from prostate cancer.

In another embodiment, the present invention provides a method of suppressing, inhibiting or reducing the risk of developing androgen-deprivation induced loss of bone mineral density (BMD) in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby suppressing, inhibiting or reducing the risk of developing androgen-deprivation induced loss of bone mineral density (BMD) in a male human subject suffering from prostate cancer.

In another embodiment, the present invention provides a method of treating androgen-deprivation induced bone fractures in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby treating androgen-deprivation induced bone fractures in a male human subject suffering from prostate cancer.

In another embodiment, the present invention provides a method of preventing androgen-deprivation induced bone fractures in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby preventing androgen-deprivation induced bone fractures in a male human subject suffering from prostate cancer.

In another embodiment, the present invention provides a method of suppressing, inhibiting or reducing the risk of developing androgen-deprivation induced bone fractures in a male human subject suffering from prostate cancer, wherein the subject has a precipitous decline in androgen levels, said method comprising the step of administering 80 mg per day of Toremifene citrate, or a pharmaceutically acceptable salt thereof to said subject, wherein said method increases bone density without increasing androgen levels in the subject, thereby suppressing, inhibiting or reducing the risk of developing androgen-deprivation induced bone fractures in a male human subject suffering from prostate cancer.

Osteoporosis is a systemic skeletal disease, characterized by low bone mass and deterioration of bone tissue, with a consequent increase in bone fragility and susceptibility to fracture. In osteoporotic patients, bone strength is abnormal, with a resulting increase in the risk of fracture. Osteoporosis depletes both the calcium and the protein collagen normally found in the bone, resulting in either abnormal bone quality or decreased bone density. Bones that are affected by osteoporosis can fracture with only a minor fall or injury that normally would not cause a bone fracture. The fracture can be either in the form of cracking (as in a hip fracture) or collapsing (as in a compression fracture of the spine). The spine, hips, and wrists are common areas of osteoporosis bone fractures, although fractures can also occur in other skeletal areas.

BMD is a measured calculation of the true mass of bone. The absolute amount of bone as measured by bone mineral density (BMD) generally correlates with bone strength and its ability to bear weight. By measuring BMD, it is possible to predict fracture risk in the same manner that measuring blood pressure can help predict the risk of stroke.

BMD in one embodiment can be measured by known bone-mineral content mapping techniques. Bone density of the hip, spine, wrist, or calcaneus may be measured by a variety of techniques. The preferred method of BMD measurement is dual-energy x-ray densitometry (DXA). BMD of the hip, antero-posterior (AP) spine, lateral spine, and wrist can be measured using this technology. Measurement at any site predicts overall risk of fracture, but information from a specific site is the best predictor of fracture at that site. Quantitative computerized tomography (QCT) is also used to measure BMD of the spine. See for example, “Nuclear Medicine: “Quantitative Procedures”. by Wahner H W, Dunn W L, Thorsen H C, et al, published by Toronto Little, Brown & Co., 1983, (see pages 107-132). An article entitled “Assessment of Bone Mineral Part 1” appeared in the Journal of Nuclear Medicine, pp 1134-1141, (1984). Another article entitled “Bone Mineral Density of The Radius” appeared in Vol. 26, No. 11, (1985) Nov. Journal of Nuclear Medicine at pp 13-39. Abstracts on the use of gamma cameras for bone-mineral content measurements are (a) S. Hoory et al, Radiology, Vol. 157(P), p. 87 (1985), and (b) C. R. Wilson et al, Radiology, Vol. 157(P), p. 88 (1985).

The present invention provides a safe and effective method for treating, preventing, suppressing, inhibiting or reducing the risk of developing androgen-deprivation induced osteoporosis and/or loss of BMD and/or bone fractures in male subjects suffering from prostate cancer, wherein the subject has a precipitous decline in androgen levels as a consequence of androgen-deprivation therapy.

Toremifene, at the doses described herein is effective at treating, suppressing or inhibiting osteopenia accompanied by bone loss. “Osteopenia” refers to decreased calcification or density of bone. This is a term which encompasses all skeletal systems in which such a condition is noted.

The invention includes the administration of “pharmaceutically acceptable salts” of Toremifene. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

The invention includes the administration of metabolites of SERMs, for example metabolites of Toremifene, such as, for example, deaminocarboxytoremifene, 4-hydroxy-N-desmethyltoremifene, N-desmethyltoremifene, Ospemifene. The invention includes the administration of any triphenylalkane derivative, or formulation thereof, for example as described in U.S. Pat. No. 4,996,225; 5,491,173 or 6,395,785; United States Patent Application Publication Number 2005187301, 2006105045 or 2005182143, all of which are incorporated by reference in their entirety.

### Pharmaceutical Compositions

In one embodiment, the methods of the present invention comprise administering a pharmaceutical composition comprising a selective estrogen receptor modulator, for example, Toremifene at a dosage which results in the delivery of 80 mg to the subject, in single dose units. The pharmaceutical composition is administered to a male human subject suffering from prostate cancer; for treating and/or preventing androgen-deprivation induced osteoporosis and/or loss of BMD; for suppressing or inhibiting androgen-deprivation induced osteoporosis and/or loss of BMD; and/or for reducing the risk of developing androgen-deprivation induced osteoporosis and/or loss of BMD in the male subject.

As used herein, “pharmaceutical composition” means a “therapeutically effective amount” of the active ingredient, i.e. Toremifene, together with a pharmaceutically acceptable carrier or diluent. A “therapeutically effective amount” as used herein refers to that amount which provides a therapeutic effect for a given condition and administration regimen.

The pharmaceutical compositions comprising Compound I, such as, for example, Toremifene can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In one embodiment of the present invention, Toremifene is formulated in a capsule. In accordance with this embodiment, the compositions of the present invention comprise, in addition to Toremifene and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, Toremifene is formulated in a composition comprising a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of a SERM, or a Compound of Formula I, also referred to herein as Compound I, as herein described, over a period of time. In one embodiments, the pellet provides for controlled release of Toremifene, over a period of time.

In another embodiment, the SERM, or a Compound of Formula I, or Toremifene can be delivered in a vesicle, such as a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g. glycerol, polyethylene glycerol), anti-oxidants (e.g. ascorbic acid, sodium metabisulfite, butylated hydroxyanisole), stabilizers (e.g. hydroxypropyl cellulose, hydroxypropylmethyl cellulose), viscosity increasing agents (e.g. carbomer, colloidal silicon dioxide, ethyl cellulose, guar gum), sweeteners (e.g. aspartame, citric acid), preservatives (e.g. Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g. poloxamers or poloxamines), coating and film forming agents (e.g. ethyl cellulose, acrylates, polymethacrylates) and/or adjuvants.

In one embodiment, the pharmaceutical compositions for use as described herein are controlled-release compositions, i.e. compositions in which Toremifene is released over a period of time after administration. Controlled- or sustained-release compositions include formulation in lipophilic depots (e.g. fatty acids, waxes, oils). In another embodiment, the composition is an immediate-release composition, i.e. a composition in which Toremifene is released immediately after administration.

In another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled-release systems are discussed in the review by Langer (Science 249:1527-1533 (1990).

The compositions may also include incorporation of the active material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention is the modification of Toremifene by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, Toremifene is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, Toremifene is converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of Toremifene are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

In some embodiments, the term “treating” includes preventative as well as disorder remitative treatment. In some embodiments, the terms “reducing”, “suppressing” and “inhibiting” have their commonly understood meaning of lessening or decreasing. In some embodiments, the term “progression” means increasing in scope or severity, advancing, growing or becoming worse. In some embodiments, the term “recurrence” means the return of a disease after a remission.

In some embodiments, the term “administering” refers to bringing a subject in contact with an anti-estrogen compound of the present invention. In some embodiments, administration can be accomplished in vitro, i.e. in a test tube, or in vivo, i.e. in cells or tissues of living organisms, for example humans.

In one embodiment, the methods of the present invention comprise administering Toremifene as the sole active ingredient. However, also encompassed within the scope of the present invention is the administration of Toremifene at a dose of about 80 mg per day, in combination with one or more therapeutic agents.

In some embodiments, the compositions of this invention comprise Compound I, for example toremifene citrate or an analog, derivative, isomer, metabolite, pharmaceutical product, pharmaceutical salt, N-oxide or hydrate or a combination thereof in combination with a bisphosphonate. In one embodiment, the bisphosphonate is alendronate, tiludroate, clodronate, pamidronate, etidronate, zoledronate, cimadronate, neridronate, minodronic acid, ibandronate, risedronate, or homoresidronate or any combination thereof. In another embodiment the bisphosphonate is alendronate [(4-amino-1-hydroxybutylidene)bis phosphonic acid, disodium salt, hydrate]. In another embodiment the bisphosphonate is clodronate [(dichloromethylene)bis phosphonic acid, disodium salt]. In another embodiment the bisphosphonate is pamidronate (3-amino-1-hydroxypropylidene)bis phosphonic acid, disodium salt). In another embodiment the bisphosphonate is risedronate (1-hydroxy-2-(3-pyridinyl)ethylidene bisphosphonic acid monosodium salt). In another embodiment the bisphosphonate is homorisedronate. In another embodiment, the bisphosphonate is tiludroate [[(4-Chlorophenyl)thio]methylene]bis[phosphonic acid], disodium salt. In another embodiment the bisphosphonate is etidronate [(1-hydroxyethylidene)bisphosphonate]. In another embodiment the bisphosphonate is zoledronate [(1-hydroxy-2-imidazol-1-yl-1-phosphono-ethyl)phosphonic acid]. In another embodiment the bisphosphonate is cimadronate [1-(cycloheptylamino)methylidene-1,1-bisphosphonic acid]. In another embodiment the bisphosphonate is neridronate [6-Amino-1-hydroxyhexylidene bisphosphonic]. In another embodiment the bisphosphonate is ibandronate [sodium is 3-(N-methyl-N-pentyl)amino-1-hydroxypropane-1,1-diphosphonic acid, monosodium salt, monohydrate]. In another embodiment the bisphosphonate is minodronic acid.

These agents include, but are not limited to: LHRH analogs, reversible antiandrogens (such as bicalutamide or flutamide), additional anti-estrogens, anticancer drugs, 5-alpha reductase inhibitors, aromatase inhibitors, progestins, selective androgen receptor modulators (SARMS) or agents acting through other nuclear hormone receptors.

Thus, in one embodiment, the methods of the present invention include using compositions and pharmaceutical compositions providing Toremifene at a dose of 80 mg per day and further comprising an LHRH analog. In another embodiment, the methods of the present invention include using compositions and pharmaceutical compositions providing Toremifene at a dose of 80 mg per day and further comprising a reversible antiandrogen. In another embodiment, the methods of the present invention include using compositions and pharmaceutical compositions providing Toremifene at a dose of 80 mg per day and further comprising an anti-estrogen. In another embodiment, the methods of the present invention include using compositions and pharmaceutical compositions providing Toremifene at a dose of 80 mg per day and further comprising with an anticancer drug. In another embodiment, the methods of the present invention include using compositions and pharmaceutical compositions providing Toremifene at a dose of 80 mg per day and further comprising a 5-alpha reductase inhibitor. In another embodiment, the methods of the present invention include using compositions and pharmaceutical compositions providing Toremifene at a dose of 80 mg per day and further comprising an aromatase inhibitor. In another embodiment, the methods of the present invention include using compositions and pharmaceutical compositions providing Toremifene at a dose of 80 mg per day and further comprising a progestin. In another embodiment, the methods of the present invention include using compositions and pharmaceutical compositions comprising providing Toremifene at a dose of 80 mg per day and further comprising a SARM. In another embodiment, the methods of the present invention include using compositions and pharmaceutical compositions providing Toremifene at a dose of 80 mg per day and further comprising an agent acting through other nuclear hormone receptors.

The following examples are presented in order to more fully illustrate the preferred embodiments of the invention. They should in no way, however, be construed as limiting the broad scope of the invention.

### EXPERIMENTAL DETAILS SECTION

### Example 1

### Effect of 80 mg Toremifene on Increasing Bone Density in a Human Clinical Trial

Men with a histologically confirmed diagnosis of prostate cancer who have been treated with ADT for at least 6 months, greater than 70 years of age or at least 50 years of age with evidence of osteopenia by baseline dual energy X-ray absorptiometry (DEXA) scan were assigned randomly to receive either toremifene citrate 80 mg daily or placebo. Treatment was continued for 12 months at which time a DEXA scan was performed.

200 men were assessed in this study.

Table 1-1 shows the age distribution of the subjects in the study.

TABLE 1-1

| | | Toremifene, | |
|---|---|---|---|
| Variable | Placebo | 80 mg | Total |
| Sample size | 104 | 93 | 197 |
| Mean | 77.5 | 76.3 | 76.9 |
| SD | 6.45 | 6.89 | 6.67 |
| Median | 79.0 | 77.0 | 78.0 |
| Minimum | 60 | 54 | 54 |
| Maximum | 90 | 89 | 90 |

Table 1-2 demonstrates mean change from baseline to month 12 in lumbar bone mineral density for subjects that have completed 12 months of treatment

TABLE 1-2

| | | | Toremifene | | |
|---|---|---|---|---|---|
| | Visit | Placebo | 80 mg | Pooled | p-value vs. |
| | Statistic | (n = 104) | (n = 93) | SD | Placebo [1] |
| Baseline | n | 104 | 93 | | |
| | Mean | 1.0388 | 1.1025 | | |
| | Median | 1.0075 | 1.0880 | | |
| | LS Mean | 1.0388 | 1.1025 | 0.20806 | 0.033* |
| | SE LS Mean | 0.02040 | 0.02157 | | |
| | Min-Max | 0.694-1.670 | 0.692-1.761 | | |
| Change from | n | 104 | 93 | | |
| Baseline to | Mean | −0.0076 | 0.0164 | | |
| Month 12 | Median | −0.0050 | 0.0100 | | |
| | LS Mean | −0.0080 | 0.0169 | 0.03492 | <0.001* |
| | SE LS Mean | 0.00344 | 0.00364 | | |
| | Min-Max | −0.122-0.060 | −0.050-0.117 | | |

*= Denotes statistical significance at the 0.045 level.
[1] P-values are from an ANOVA with treatment as the factor at the Baseline Visit and from an ANOVA with treatment and baseline BMD as the factors at the Month 12 Visit.

Table 1-3 shows mean change from baseline to month 12 in the lumbar bone mineral density for subjects that have completed 12 months of treatment, including the site.

TABLE 1-3

| | | | Toremifene | | |
|---|---|---|---|---|---|
| | Visit | Placebo | 80 mg | Pooled | p-value vs. |
| | Statistic | (n = 104) | (n = 93) | SD | Placebo [1] |
| Baseline | n | 104 | 93 | | |
| | Mean | 1.0388 | 1.1025 | | |
| | Median | 1.0075 | 1.0880 | | |
| | LS Mean | 1.0500 | 1.0972 | 0.18527 | 0.099 |
| | SE LS Mean | 0.02125 | 0.02367 | | |
| | Min-Max | 0.694-1.670 | 0.692-1.761 | | |
| Change from | n | 104 | 93 | | |
| Baseline to | Mean | 0.0076 | 0.0164 | | |
| Month 12 | Median | 0.0050 | 0.0100 | | |
| | LS Mean | 0.0044 | 0.0193 | 0.03452 | <0.001* |
| | SE LS Mean | 0.00397 | 0.00443 | | |
| | Min-Max | −0.122-0.060 | −0.050-0.117 | | |

Table 1-4 summarizes the percent change from baseline in lumbar bone mineral density, for subjects who have completed 12 months of treatment.

TABLE 1-4

| | Visit | | Toremifene | Pooled |
|---|---|---|---|---|
| | Statistic | Placebo | 80 mg | SD |
| Baseline | n | 104 | 93 | |
| | Mean | 1.039 | 1.102 | 0.2100 |
| | SD | 0.1935 | 0.2233 | |
| | Median | 1.008 | 1.088 | |
| | LS Mean | 1.0500 | 1.0972 | 0.18527 |
| | Min-Max | 0.69-1.670 | 0.69-1.76 | |
| Change from | n | 104 | 93 | |
| Baseline to | Mean | −0.694 | 1.593 | |
| Month 12 | SD | 3.2742 | 3.4025 | |
| | Median | −0.4555 | 0.960 | |
| | Min-Max | −12.13-6.51 | −5.02-9.80 | |

Table 1-5 summarizes mean changes from baseline to month 12 in femur bone mineral density.

TABLE 1-5

| | | | Toremifene | Pooled | p-value vs. |
|---|---|---|---|---|---|
| | Statistic | Placebo | 80 mg | SD | Placebo [1] |
| Baseline | n | 103 | 92 | | |
| | Mean | 0.7636 | 0.8033 | | |
| | Median | 0.7570 | 0.7760 | | |
| | LS Mean | 0.7636 | 0.8033 | 0.15258 | 0.071 |
| | SE LS Mean | 0.01503 | 0.01591 | | |
| | Min-Max | 0.515-1.114 | 0.528-1.198 | | |
| Changes from | n | 103 | 92 | | |
| Baseline at | Mean | −0.0103 | 0.0013 | | |
| month 12 | Median | −0.0090 | 0.0030 | | |
| | LS Mean | −0.0105 | 0.0016 | 0.03145 | 0.009* |
| | SE LS Mean | 0.00311 | 0.00329 | | |
| | Min-Max | −0.120-0.064 | −0.132-0.077 | | |

Table 1-6 summarizes mean changes from baseline to month 12 in femur bone mineral density, including the site.

TABLE 1-6

| | | | Toremifene | Pooled | p-value vs. |
|---|---|---|---|---|---|
| | Statistic | Placebo | 80 mg | SD | Placebo [1] |
| Baseline | n | 103 | 92 | | |
| | Mean | 0.7636 | 0.8033 | | |
| | Median | 0.7570 | 0.7760 | | |
| | LS Mean | 0.7756 | 0.7833 | 0.13216 | 0.710 |
| | SE LS Mean | 0.01539 | 0.01708 | | |
| | Min-Max | 0.515-1.114 | 0.528-1.198 | | |
| Change from | n | 103 | 92 | | |
| Baseline to | Mean | −0.0103 | 0.0013 | | |
| Month 12 | Median | −0.0090 | 0.0030 | | |
| | LS Mean | −0.0125 | 0.0014 | 0.03168 | 0.006* |
| | SE LS Mean | 0.00369 | 0.00409 | | |
| | Min-Max | −0.120-0.064 | −0.132-0.077 | | |

Table 1-7 shows the percentage change from baseline to month 12 in femur bone mineral density.

TABLE 1-7

| | | | Toremifene | |
|---|---|---|---|---|
| | Statistic | Placebo | 80 mg | Pooled SD |
| Baseline | n | 103 | 92 | |
| | Mean | 0.764 | 0.803 | 0.1535 |
| | SD | 0.1346 | 0.1705 | |
| | Median | 0.757 | 0.776 | |
| | Min-Max | 0.52-1.11 | 0.53-1.20 | |
| Percentage | n | 103 | 92 | |
| Change from | Mean | −1.302 | 0.173 | |
| Baseline to | SD | 4.2801 | 3.6716 | 4.0621 |
| Month 12 | Median | −1.190 | 0.505 | |
| | Min-Max | −18.13-8.38 | −13.02-10.91 | |

Table 1-8 shows the mean change from baseline to month 12 in the hip bone mineral density for subjects that have completed 12 months of treatment.

TABLE 1-8

| | | | Toremifene | Pooled | p-value vs. |
|---|---|---|---|---|---|
| | Statistic | Placebo | 80 mg | SD | Placebo [1] |
| Baseline | n | 103 | 91 | | |
| | Mean | 0.8917 | 0.9181 | | |
| | Median | 0.8900 | 0.8950 | | |
| | LS Mean | 0.8917 | 0.9181 | 0.15248 | 0.231 |
| | SE LS Mean | 0.01502 | 0.01598 | | |
| | Min-Max | 0.602-1.257 | 0.608-1.331 | | |
| Change from | n | 103 | 91 | | |
| Baseline to | Mean | −0.0112 | 0.0049 | | |
| Month 12 | Median | −0.0100 | 0.0040 | | |
| | LS Mean | −0.0113 | 0.0050 | 0.03313 | |
| | SE LS Mean | 0.00327 | 0.00348 | | 0.001* |
| | Min-Max | −0.131-0.105 | −0.083-0.290 | | |

Table 1-9 shows mean change from baseline to month 12 in the hip bone mineral density, including the site.

TABLE 1-9

| | | | Toremifene | Pooled | p-value vs. |
|---|---|---|---|---|---|
| | Statistic | Placebo | 80 mg | SD | Placebo [1] |
| Baseline | n | 103 | 91 | | |
| | Mean | 0.8917 | 0.9181 | | |
| | Median | 0.8900 | 0.8950 | | |
| | LS Mean | 0.8914 | 0.8934 | 0.14733 | 0.931 |
| | SE LS Mean | 0.01716 | 0.01914 | | |
| | Min-Max | 0.602-1.257 | 0.608-1.331 | | |
| Change from | n | 103 | 91 | | |
| Baseline to | Mean | −0.0112 | 0.0049 | | |
| Month 12 | Median | −0.0100 | 0.0040 | | |
| | LS Mean | −0.0137 | 0.0049 | 0.03459 | 0.001* |
| | SE LS Mean | 0.00404 | 0.00450 | | |
| | Min-Max | −0.131-0.105 | −0.083-0.290 | | |

Table 1-10 shows the percentage change from baseline to month 12 in the hip bone mineral density, including the site.

TABLE 1-10

| | | | Toremifene | Pooled |
|---|---|---|---|---|
| | Statistic | Placebo | 80 mg | SD |
| Baseline | n | 103 | 91 | |
| | Mean | 0.892 | 0.918 | 0.1527 |
| | SD | 0.1364 | 0.1688 | |
| | Median | 0.890 | 0.895 | |
| | Min-Max | 0.60-1.26 | 0.61-1.33 | |
| Percentage | n | 103 | 91 | |
| Change from | Mean | −1.284 | 0.672 | 4.4508 |
| Baseline to | SD | 3.0961 | 5.4371 | |
| Month 12 | Median | −1.210 | 0.420 | |
| | Min-Max | −12.93-8.35 | −6.65-47.31 | |

Cumulative DEXA results compared to baseline were as summarized in Table 1-11 below:

TABLE 1-11

| | Toremifene | | | |
|---|---|---|---|---|
| | 80 mg (% | Placebo (% | | |
| | change from | change from | Treatment | |
| | baseline) | baseline) | Effect (%) | p value |
| Lumbar | 1.6 | −0.69 | 2.3 | <0.001 |
| Spine | | | | |
| Total Hip | 0.67 | −1.3 | 2.0 | 0.001 |
| Femoral | 0.17 | −1.3 | 1.5 | 0.009 |
| Neck | | | | |

Toremifene citrate produced statistically significant and clinically meaningful changes in bone mineral density in men treated with ADT for prostate cancer. A clinically meaningful decrease in BMD was apparent in the placebo group confirming the occurrence of accelerated BMD loss in men treated with ADT. The magnitude of BMD preservation and increase seen in men treated with toremifene citrate was similar to that seen in clinical trials with SERMs, in treating reductions in fracture rates in post-menopausal women. Toremifene citrate thus will provide a fracture reduction benefit.

### Example 2

### Effect of 80 mg Toremifene on Diminishing Bone Loss or Bone Fractures in a Phase III Human Clinical Trial

A human clinical trial with 1,389 male subjects having undergone ADT was conducted. Subjects were randomized into the double-blinded study to evaluate treatment with toremifene citrate (80 mg) compared to placebo over a course of two years at approximately 150 clinical sites in the United States and Mexico. The primary endpoint was new morphometric vertebral fractures read by an independent third party.

Subjects were treated with 80 mg of Toremifene citrate daily. The presence of new morphometric vertebral fractures was evaluated in Toremifene- and placebo-treated men. The following Table 2-1 describes the number of newly developed fractures in the subjects:

TABLE 2-1

| Population | Placebo | Treated |
|---|---|---|
| MITT | 24 | 11 |
| MITT w/NOPs | 24 | 12 |
| MITT w/NOP | 25 | 12 |
| Eff Eval | 22 | 8 |
| Eff Eval w/NOPs | 22 | 9 |

** MITT - subjects that had at least one on study radiograph, new morphometric vertebral fractures; Eff Eval (Efficacy Evaluable) - subjects in the MITT that were compliant with the protocol (didn't take prohibited meds, took >80% of their study medication, were maintained on castration, etc.); NOPs - “non-osteoporotic” fracture, change in the shape of the vertebrae is due to bone metastasis

The values obtained for Toremifene versus placebo treated subjects for each indicated population evaluated was compared.

Table 2-2 describes the percent of subjects which exhibited new morphometric vertebral fractures:

TABLE 2-2

| Population | Placebo FX | Treated FX | % Reduction | p-value |
|---|---|---|---|---|
| MITT | 4.9% | 2.3% | 53% | 0.032 |
| w/NOP | 5.1% | 2.5% | 51% | 0.038 |

Table 2-3 describes the number of new morphometric vertebral fracture in subjects having received at least one dose of either Toremifene or placebo:

TABLE 2-3

| Population | Placebo FX | Treated FX | % Reduction | p-value |
|---|---|---|---|---|
| ITT | 3.5% | 1.6% | 54% | 0.023 |
| w/NOPs | 3.6% | 1.7% | 53% | 0.027 |

The above ITT population represents a population for addressing the safety of the administration of the compound.

Table 2-4 describes the percent of subjects exhibiting new fractures in Toremifene-treated versus placebo treated groups, who were compliant with the treatment regimen:

TABLE 2-4

| Population | Placebo FX | Treated FX | % Reduction | p-value |
|---|---|---|---|---|
| Eff Eval | 5.1% | 2.0% | 61% | 0.017 |
| w/NOPs | 5.3% | 2.3% | 58% | 0.022 |

The time to event, in terms of the formation of new morphometric vertebral fractures in treated subjects, who were treated with a relatively complete regimen versus inclusion of subjects having taken an incomplete regimen showed a significant difference in comparison to placebo treated individuals Table 2-5 describes the statistical significance in terms of time to event in treated groups, where the significance is versus the placebo group:

TABLE 2-5

| | P-value |
|---|---|
| MITT w/NOP | 0.062 |
| ITT w/NOP | 0.062 |
| MITT | 0.043 |
| ITT | 0.045 |

Thus, Toremifene citrate 80 mg demonstrated a 53% reduction in new morphometric vertebral fractures (p=0.034; 3.6% fracture rate in the placebo group) in an intent to treat analysis among all patients randomized into the trial. Toremifene citrate 80 mg demonstrated a 50% reduction in morphometric vertebral fractures (p<0.05; 5.0% fracture rate in the placebo group) in a modified intent to treat analysis, which included patients with at least one evaluable study radiograph and a minimum of one dose of study drug or placebo. In pre-specified subset analyses, among patients who were greater than 80% treatment compliant, toremifene citrate 80 mg reduced vertebral morphometric fractures by 61% (p=0.017). When patients who experienced greater than 7% bone loss at one year were considered along with new morphometric vertebral fractures as treatment failures, toremifene citrate 80 mg compared to placebo demonstrated a 56% reduction (p=0.003).

In addition to effects of Toremifene on reducing the number of new fractures in ADT-treated subjects, bone density was positively affected as well.

Table 2-6 describes bone mineral density difference from placebo in subjects with at least one fracture and having received at least one dose of Toremifene with the prescribed treatment regimen.

TABLE 2-6

| | % different from | |
|---|---|---|
| | placebo | p-value |
| MITT Spine | 1.97% | <0.0001 |
| MITT Total Hip | 1.57% | <0.0001 |
| MITT Femur | 1.64% | <0.0001 |
| ITT Spine | 1.42% | <0.0001 |
| IIT Total Hip | 1.15% | <0.0001 |
| ITT Femur | 1.18% | <0.0001 |

The placebo subjects continued to lose bone over the 24 month period evaluated. Virtually all BMD increases seen in the Toremifene group were noted in the first 12 months of the study, with no significant increases noted from month 12 to month 24, indicating Toremifene effects on stimulating bone density increases are rapid in onset and prolonged, in terms of the lack of finding diminishment in the bone density over time, unlike the placebo-treated group.

Thus, subjects treated with toremifene citrate 80 mg demonstrated statistically significant increases compared to placebo in bone mineral density in the lumbar spine (+2.0%; p<0.0001), and other skeletal sites (hip and femur) had similar increases (p<0.0001).

Five subjects treated with Toremifene as opposed to 14 placebo-treated subjects exhibited more than a seven percent bone loss by the 12 month date post ADT-treatment.

When results regarding formation of new morphometric vertebral fractures and the presence of greater than seven percent bone loss at 12 months is jointly evaluated, the results give a measure of the treatment failure, presented in the Table 2-7.

TABLE 2-7

| | % reduction | p-value |
|---|---|---|
| MITT | 53% | <0.006 |
| ITT | 56% | 0.003 |

Similarly, the worsening of new morphometric vertebral fractures was assessed in Toremifene-treated versus placebo-treated subjects, and were as presented in Table 2-8.

TABLE 2-8

| | % reduction | p-value |
|---|---|---|
| MITT | 46% | 0.065 |
| ITT | 49% | 0.044 |

Other effects on bone were assessed as described in Table 2-9.

TABLE 2-9

| | Toremifene | Placebo | % reduction | p-value |
|---|---|---|---|---|
| CFF | 27 | 31 | 14% | 0.558 |
| CFF + MVF | | | 30% | 0.087 |
| CFF + MVF + > | | | 39% | 0.01 |
| 7% bone loss* | | | | |

CFF—Clinical fragility fractures; MVF—morphometric vertebral fractures

The reduction of clinical fragility fractures, morphometric vertebral fractures and greater than seven percent bone loss when evaluated cumulatively showed a significant reduction in these bone-related events in Toremifene-versus placebo-treated subjects.

Taken together, these results indicate that Toremifene treatment surprisingly, significantly reduced bone loss and bone fractures in subjects having undergone ADT.

### Example 3

### Effects of Toremifene on Other Pathologies Associated with ADT Therapy in Human Subjects

In addition to bone loss and bone fractures, ADT is associated with the development of other pathologies, such as gynecomastia. In the clinical trial described in Example 3, subjects were treated with Toremifene as described, and the amelioration of gynecomastia as a result of treatment was evaluated.

Table 3-1 indicates incidence of pain due to gynecomastia at the indicated times post treatment. Significantly fewer individuals experienced pain from gynecomastia by the end of the study period. Moreover, while numerous placebo-treated subjects indicated a worsening of pain with time, the Toremifene-treated group minimally indicated such worsening.

TABLE 3-1

| Time | Toremifene | Placebo | p-value |
|---|---|---|---|
| 3 month | 0.02 | 0 | 0.232 |
| 6 month | 0.01 | −0.01 | 0.151 |
| End of study | 0.03 | −0.03 | 0.003 |

In addition to gynecomastia, another effect of ADT therapy is an altering of lipid profiles in treated subjects. Table 3-2 describes total cholesterol in subjects, represented as changes in percent over baseline values.

TABLE 3-2

| | Toremifene | Placebo | p-value |
|---|---|---|---|
| 12 months | −6.85 ± 15.13 | −2.87 ± 15.6 | <0.001 |
| 24 months | −7.54 ± 20.69 | −4.52 ± 17.55 | 0.011 |

Table 3-3 describes the absolute change from baseline.

TABLE 3-3

| | Toremifene | Placebo | p-value |
|---|---|---|---|
| 12 months | −15.67 ± 28.97 | −7.83 ± 31.79 | <0.001 |
| 24 months | −17.75 ± 31.83 | −11.47 ± 34.75 | 0.002 |

Similarly, changes in low density lipoprotein (LDL) were evaluated. Table 3-4 describes the percent change in LDL over baseline.

TABLE 3-4

| | Toremifene | Placebo | p-value |
|---|---|---|---|
| 12 months | −9.98 ± 24.05 | −4.39 ± 23.35 | <0.001 |
| 24 months | −11.93 ± 28.49 | −7.96 ± 25.15 | 0.018 |

Table 3-5 describes the absolute change in LDL over baseline.

TABLE 3-5

| | Toremifene | Placebo | p-value |
|---|---|---|---|
| 12 months | −14.22 ± 25.25 | −7.58 ± 26.71 | <0.001 |
| 24 months | −16.22 ± 26.46 | −11.95 ± 28.40 | 0.012 |

Changes in high density lipoprotein values were evaluated as well. Table 3-6 describes the percent change in HDL over baseline.

TABLE 3-6

| | Toremifene | Placebo | p-value |
|---|---|---|---|
| 12 months | 5.98 ± 18.13 | 0.91 ± 17.7 | <0.0001 |
| 24 months | 6.74 ± 22.76 | 2.55 ± 19.56 | 0.001 |

Table 3-7 describes the absolute change in HDL over baseline.

TABLE 3-7

| | Toremifene | Placebo | p-value |
|---|---|---|---|
| 12 months | 2.44 ± 8.77 | −0.23 ± 8.16 | <0.0001 |
| 24 months | 2.56 ± 9.55 | 0.64 ± 8.59 | <0.001 |

Changes in triglyceride levels were evaluated as well. Table 3-8 describes the percent change in triglycerides over baseline.

TABLE 3-8

| | Toremifene | Placebo | p-value |
|---|---|---|---|
| 12 months | −6.59 ± 33.94 | 10.51 ± 59.65 | <0.0001 |
| 24 months | −4.86 ± 41.44 | 9.91 ± 57.75 | <0.0001 |

Table 3-9 describes the absolute change in triglycerides over baseline.

TABLE 3-9

| | Toremifene | Placebo | p-value |
|---|---|---|---|
| 12 months | −21.21 ± 62.0 | −1.62 ± 80.79 | 0.0001 |
| 24 months | −19.72 ± 70.88 | −1.79 ± 89.28 | <0.001 |

Toremifene citrate 80 mg treatment compared to placebo also resulted in a decrease in total cholesterol (p=0.011), LDL (p=0.018), and triglycerides (p<0.0001), and an increase in HDL (p=0.001). Taken together, the results show a Toremifene-mediated reduction in cholesterol, LDL and triglyceride levels and a pronounced increase in HDL levels, thus optimally altering lipid profiles in treated subjects.

It will be appreciated by a person skilled in the art that the present invention is not limited by what has been particularly shown and described hereinabove. Rather, the scope of the invention is defined by the claims which follow:

What is claimed is:

1. A method of treating androgen-deprivation induced osteoporosis in a male human subject suffering from prostate cancer, said method comprising the step of administering 80 mg per day of a compound of Formula I: 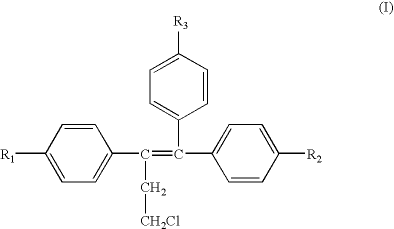
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby treating androgen-deprivation induced osteoporosis in a male human subject suffering from prostate cancer.

2. The method according to claim 1, wherein said compound is Toremifene citrate.

3. The method according to claim 2, wherein said administering comprises administering a pharmaceutical composition comprising said Toremifene and/or its isomer, metabolite, pharmaceutically acceptable salt, pharmaceutical product, hydrate, N-oxide, or any combination thereof; and a pharmaceutically acceptable carrier.

4. The method according to claim 3, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

5. The method according to claim 3 wherein said pharmaceutical composition is a pellet, a tablet, a capsule, a solution, a suspension, an emulsion, an elixir, a gel, a cream, a suppository or a parenteral formulation.

6. A method of preventing androgen-deprivation induced osteoporosis in a male human subject suffering from prostate cancer, said method comprising the step of administering 80 mg per day of a compound of Formula I: 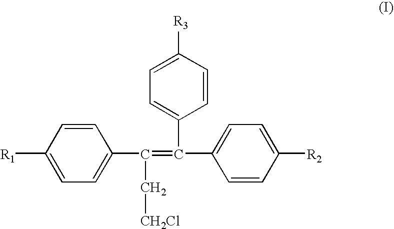
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby preventing androgen-deprivation induced osteoporosis in a male human subject suffering from prostate cancer.

7. The method according to claim 6, wherein said compound is Toremifene citrate.

8. The method according to claim 7, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

9. The method according to claim 8, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

10. The method according to claim 8 wherein said pharmaceutical composition is a pellet, a tablet, a capsule, a solution, a suspension, an emulsion, an elixir, a gel, a cream, a suppository or a parenteral formulation.

11. A method of suppressing, inhibiting, reducing the incidence or severity of, or reducing the risk of developing androgen-deprivation induced osteoporosis in a male human subject suffering from prostate cancer, wherein the subject has a precipitous decline in androgen levels, said method comprising the step of administering 80 mg per day of a compound of Formula I: 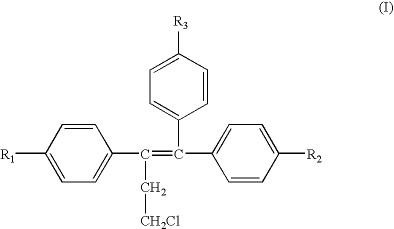
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby suppressing, inhibiting, reducing the incidence or severity of, or reducing the risk of developing androgen-deprivation induced osteoporosis in a male human subject suffering from prostate cancer

12. The method of claim 11, wherein said compound is Toremifene citrate.

13. The method according to claim 12, wherein said administering comprises administering a pharmaceutical composition comprising said Toremifene or its, isomer, metabolite, pharmaceutically acceptable salt, pharmaceutical product, hydrate, N-oxide, or any combination thereof; and a pharmaceutically acceptable carrier.

14. The method according to claim 13, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

15. The method according to claim 13 wherein said pharmaceutical composition is a pellet, a tablet, a capsule, a solution, a suspension, an emulsion, an elixir, a gel, a cream, a suppository or a parenteral formulation.

16. A method of treating androgen-deprivation induced loss of bone mineral density (BMD) in a male human subject suffering from prostate cancer, wherein the subject has a precipitous decline in androgen levels, said method comprising the step of administering 80 mg per day of a compound of Formula I: 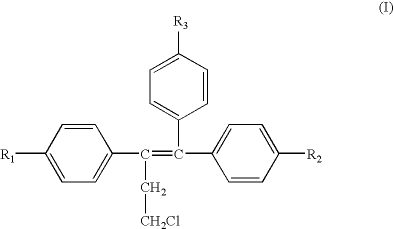
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby treating androgen-deprivation induced loss of bone mineral density (BMD) in a male human subject suffering from prostate cancer.

17. The method according to claim 16, wherein said compound is Toremifene citrate.

18. The method according to claim 17, wherein said administering comprises administering a pharmaceutical composition comprising said Toremifene or its isomer, metabolite, pharmaceutically acceptable salt, pharmaceutical product, hydrate, N-oxide, or any combination thereof; and a pharmaceutically acceptable carrier.

19. The method according to claim 18, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

20. The method according to claim 18 wherein said pharmaceutical composition is a pellet, a tablet, a capsule, a solution, a suspension, an emulsion, an elixir, a gel, a cream, a suppository or a parenteral formulation.

21. A method of preventing androgen-deprivation induced loss of bone mineral density (BMD) in a male human subject suffering from prostate cancer, wherein the subject has a precipitous decline in androgen levels, said method comprising the step of administering 80 mg per day of a compound of Formula I: 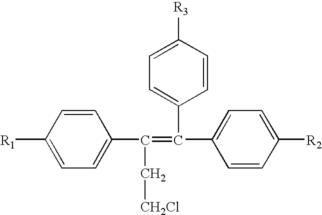
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby preventing androgen-deprivation induced loss of bone mineral density (BMD) in a male human subject suffering from prostate cancer. The method according to claim 21, wherein said compound is Toremifene citrate.

22. The method according to claim 22, wherein said administering comprises administering a pharmaceutical composition comprising said Toremifene and/or its analog, derivative, isomer, metabolite, pharmaceutically acceptable salt, pharmaceutical product, hydrate, N-oxide, or any combination thereof; and a pharmaceutically acceptable carrier.

23. The method according to claim 23, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

24. The method according to claim 23 wherein said pharmaceutical composition is a pellet, a tablet, a capsule, a solution, a suspension, an emulsion, an elixir, a gel, a cream, a suppository or a parenteral formulation.

25. A method of suppressing, inhibiting or reducing the risk of developing androgen-deprivation induced loss of bone mineral density (BMD) in a male human subject suffering from prostate cancer, wherein the subject has a precipitous decline in androgen levels, said method comprising the step of administering 80 mg per day of a compound of Formula I: 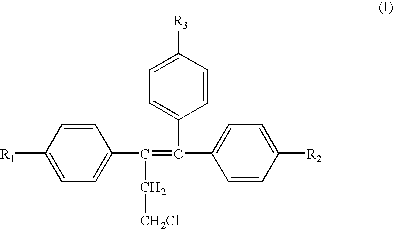
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby suppressing, inhibiting or reducing the risk of developing androgen-deprivation induced loss of bone mineral density (BMD) in a male human subject suffering from prostate cancer.

26. The method according to claim 26, wherein said compound is Toremifene citrate.

27. The method according to claim 27, wherein said administering comprises administering a pharmaceutical composition comprising said Toremifene or its isomer, metabolite, pharmaceutically acceptable salt, pharmaceutical product, hydrate, N-oxide, or any combination thereof; and a pharmaceutically acceptable carrier.

28. The method according to claim 28, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

29. The method according to claim 28 wherein said pharmaceutical composition is a pellet, a tablet, a capsule, a solution, a suspension, an emulsion, an elixir, a gel, a cream, a suppository or a parenteral formulation.

30. A method of treating androgen-deprivation induced bone fractures in a male human subject suffering from prostate cancer, wherein the subject has a precipitous decline in androgen levels, said method comprising the step of administering 80 mg per day of a compound of Formula I: 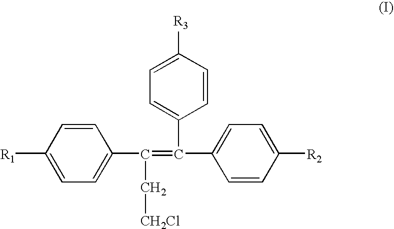
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby treating androgen-deprivation induced bone fractures in a male human subject suffering from prostate cancer.

31. The method according to claim 31, wherein said compound is Toremifene citrate.

32. The method according to claim 32, wherein said administering comprises administering a pharmaceutical composition comprising said Toremifene or its isomer, metabolite, pharmaceutically acceptable salt, pharmaceutical product, hydrate, N-oxide, or any combination thereof; and a pharmaceutically acceptable carrier.

33. The method according to claim 33, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

34. The method according to claim 33 wherein said pharmaceutical composition is a pellet, a tablet, a capsule, a solution, a suspension, an emulsion, an elixir, a gel, a cream, a suppository or a parenteral formulation.

35. A method of preventing androgen-deprivation induced bone fractures in a male human subject suffering from prostate cancer, wherein the subject has a precipitous decline in androgen levels, said method comprising the step of administering 80 mg per day of a compound of Formula I: 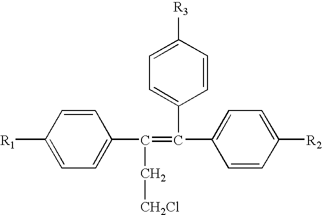
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby preventing androgen-deprivation induced bone fractures in a male human subject suffering from prostate cancer.

36. The method of claim 36, wherein said compound is Toremifene citrate.

37. The method according to claim 37, wherein said administering comprises administering a pharmaceutical composition comprising said Toremifene or its isomer, metabolite, pharmaceutically acceptable salt, pharmaceutical product, hydrate, N-oxide, or any combination thereof; and a pharmaceutically acceptable carrier.

38. The method according to claim 38, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

39. The method according to claim 38 wherein said pharmaceutical composition is a pellet, a tablet, a capsule, a solution, a suspension, an emulsion, an elixir, a gel, a cream, a suppository or a parenteral formulation.

40. A method of suppressing, inhibiting or reducing the risk of developing, or reducing the incidence or severity of androgen-deprivation induced bone fractures in a male human subject suffering from prostate cancer, wherein the subject has a precipitous decline in androgen levels, said method comprising the step of administering 80 mg per day of a compound of Formula I: 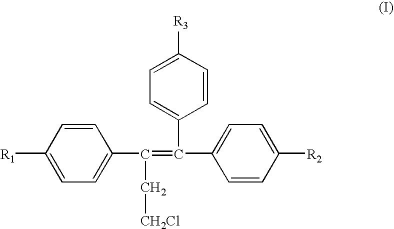
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms or its isomer, metabolite, pharmaceutically acceptable salt, pharmaceutical product, hydrate, N-oxide, or any combination thereof to said subject, wherein said method increases bone density without increasing testosterone levels in the subject, thereby suppressing, inhibiting or reducing the risk of developing, or reducing the incidence or severity of androgen-deprivation induced bone fractures in a male human subject suffering from prostate cancer.

41. The method according to claim 41, wherein said compound is Toremifene citrate.

42. The method according to claim 42, wherein said administering comprises administering a pharmaceutical composition comprising said anti-estrogen and/or its analog, derivative, isomer, metabolite, pharmaceutically acceptable salt, pharmaceutical product, hydrate, N-oxide, or any combination thereof; and a pharmaceutically acceptable carrier.

43. The method according to claim 43, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

44. The method according to claim 43 wherein said pharmaceutical composition is a pellet, a tablet, a capsule, a solution, a suspension, an emulsion, an elixir, a gel, a cream, a suppository or a parenteral formulation.
